(19) United States
(12) Patent Application Publication
(10) Pub. No.: US 2021/0174966 A1
(43) Pub. Date: Jun. 10, 2021

(54) PREDICTING AND PREVENTING HYPOGLYCEMIA IN PATIENTS HAVING TYPE 1 DIABETES DURING PERIODS OF INCOGNIZANCE USING BIG DATA ANALYTICS AND DECISION THEORETIC ANALYSIS

(71) Applicant: Oregon Health & Science University, Portland, OR (US)

(72) Inventors: Peter G. Jacobs, Portland, OR (US); Clara Mosquera-Lopez, Portland, OR (US); Nichole Tyler, Portland, OR (US); Robert Dodier, Portland, OR (US); Jessica Castle, Portland, OR (US); Joseph E. Youssef, Portland, OR (US); Leah Wilson, Portland, OR (US)

(21) Appl. No.: 17/114,433

(22) Filed: Dec. 7, 2020

Related U.S. Application Data
(60) Provisional application No. 62/944,287, filed on Dec. 5, 2019.

(51) Int. Cl. G16H 50/30; G16H 20/60; G16H 20/17; G16H 20/30; G16H 10/60; G16H 50/50; G06N 3/08

(52) U.S. Cl. CPC G16H 50/30; G16H 20/60; G16H 20/17; G06N 3/08; G16H 10/60; G16H 50/50; G16H 20/30

(57) ABSTRACT

Disclosed are techniques for supporting glucoregulatory management decisions using a decision support recommender to predict an occurrence of a hypoglycemic event in an incognizant living subject. Glucose management data is obtained from a living subject. It is processed as prescribed by a feature extractor to generate a set of glucoregulatory feature values. The glucoregulatory feature values are applied as prescribed by the feature extractor to a hypoglycemia prediction model. The hypoglycemia prediction model is formulated to predict the occurrence of a hypoglycemic event during a period of incognizance of the living subject. A glucoregulatory management recommendation is generated as prescribed by the decision support recommender if the hypoglycemia prediction model predicts the occurrence of a hypoglycemic event during the period of incognizance.

### RELATED APPLICATION

This application claims priority benefit of U.S. Provisional Patent Application No. 62/944,287, filed Dec. 5, 2019, which is hereby incorporated by reference in its entirety.

### FEDERALLY SPONSORED RESEARCH OR DEVELOPMENT

This invention was made with government support under DK120367 awarded by the National Institutes of Health. The government has certain rights in the invention.

### TECHNICAL FIELD

This disclosure relates to predicting and preventing hypoglycemia during periods of incognizance, including nocturnal hypoglycemia, (for example, glucose less than 70 mg/dL) in type 1 diabetics and, in particular, to use of machine learning algorithms trained on a big data set to help patients with type 1 diabetes anticipate and potentially avoid hypoglycemia by notifying them of their risk before going to sleep.

### BACKGROUND INFORMATION

People with type 1 diabetes (T1D) require lifelong exogenous insulin treatment to maintain adequate blood glucose control; however, intensive glucose control increases the risk of level 2 hypoglycemic episodes (<54 mg/dL) that result in seizures and potentially fatal consequences. Despite the advances in insulin delivery technologies including sensor-augmented insulin-pumps and artificial pancreas systems, hypoglycemia still constitutes a barrier to achieving improved glycemic control. Fear of hypoglycemia can lead to poor glucose management decisions such as insulin under dosing, or extra intake of carbohydrates. For example, nocturnal hypoglycemia, which accounts for 55% of level 2 hypoglycemia events in patients with T1D and 75% of level 2 events in children, is particularly risky because patients are unlikely to recognize symptoms while sleeping or awaken in response to hypoglycemia alarms from continuous glucose monitors. In addition to the serious short-term effects of nocturnal hypoglycemia episodes, untreated nocturnal hypoglycemia can further impair the physiological counter-regulatory system and contribute to hypoglycemia unawareness, which may eventually result in recurrent asymptomatic hypoglycemia and in some cases the dead in bed syndrome caused by prolonged exposure to extremely low blood glucose levels during sleep.

Prediction of nocturnal hypoglycemia at bedtime has been less studied than hypoglycemia in patients with T1D. Algorithms for short-term hypoglycemia prediction have been used in single- and dual-hormone automated insulin delivery systems (i.e., artificial pancreas) and proven to be effective in reducing the occurrence and duration of nocturnal hypoglycemia. Although such delivery systems are effective at improving glycemic control, the majority of patients with T1D continue to manage their glucose using multiple daily insulin injection (MDI) therapy. Without the benefit of automated insulin delivery and glucose sensing, patients using MDI must be proactive about adjusting their insulin or consuming a carbohydrate before bed to prevent nocturnal hypoglycemia.

Thus far, only Sakurai et al. report a technique for predicting nocturnal hypoglycemia prior to bedtime, but this algorithm was designed for people with type 2 diabetes. (Sakurai K, Kawai Y, Yamazaki M et al., Prediction of lowest nocturnal blood glucose level based on self-monitoring of blood glucose in Japanese patients with type 2 diabetes, J. Diabetes Complications (2018)). Sakurai et al. proposed a mathematical model to predict the lowest nocturnal glucose in patients with insulin-treated type 2 diabetes. The authors showed that the linear combination of age, fasting blood glucose level, and daily basal insulin dose could predict the lowest nocturnal glucose concentration within the A and B regions of the Clarke error grid with a root-mean-square-error of about 31 mg/dL. This prediction model was developed and validated on a small dataset.

Increasing use of continuous glucose monitor (CGM) technology and smart insulin delivery devices has resulted in growing data availability that can be used to train machine learning algorithms. (See e.g., Beam A L and Kohane I S, Big data and machine learning in healthcare, JAMA 2018; 319 (13): 1317-1318 and Zhu L and Zheng W J, Informatics, Data Science, and Artificial Intelligence, JAMA 2018, 320(ii): 1103-1104). Beam et al. report that large datasets provide an opportunity to leverage machine learning and big data analytics methods to develop robust data-driven models that can be employed in T1D to anticipate and help prevent glucose excursions. Despite new glucose sensing technologies, nocturnal hypoglycemia is difficult to prevent and treat because patients may not have symptoms or respond to sensor alarms while sleeping.

### SUMMARY OF THE DISCLOSURE

This disclosure relates to optimizing and validating a support vector regression (SVR) model that is to be used to predict and ultimately prevent nocturnal hypoglycemia by notifying a patient at bedtime to take action if there is substantial risk of nocturnal hypoglycemia. A big dataset collected from 124 patients (27,466 days) under free-living conditions was used to train and evaluate a nocturnal hypoglycemia alerting algorithm intended to provide a benefit of an accurate nocturnal hypoglycemia prediction and reduce the cost of an inaccurately predicted event using decision theory.

An SVR model was trained to predict the lowest nocturnal glucose concentration (i.e., nocturnal hypoglycemia) for people with type 1 diabetes prior to bedtime, and its operation point was derived via a decision theoretic criterion. The algorithm was trained on the dataset of 27,466 days of continuous glucose measurement (i.e., a glucose measurement taken every five minutes) and insulin data collected from 124 patients with type 1 diabetes. Features from continuous glucose monitoring data and insulin data were calculated, and the most relevant descriptors were used as inputs to the model. The threshold for announcing the risk of nocturnal hypoglycemia to the patient was derived from the SVR model output by applying a decision theoretic criterion to enhance the expected net benefit of the announcement. A secondary validation set was obtained from 10 patients with type 1 diabetes during a 4-week trial under free-living sensor-augmented insulin-pump therapy.

Primary outcome measures to assess accuracy of the algorithm are sensitivity and specificity of nocturnal hypoglycemia prediction. Secondary outcome measures are the correlation between predicted and actual minimum nocturnal glucose and root-mean-square error. On the validation dataset of 10 patients (age 34±6 years, 6F, and 18±10 years since diagnosis), the sensitivity of the algorithm in predicting nocturnal hypoglycemia events (<70 mg/dL, 95% CI, 71.3-99.9) was 94.1% and the specificity was 71.0%. Correlation between predicted and actual minimum nocturnal glucose values was R=0.73 (P<0.001). The root-mean-square error was 34.5 mg/dL. The algorithm achieved an area under the receiver operating characteristic (ROC) curve of 0.88 (95% CI, 0.77-0.99) for predicting nocturnal hypoglycemia. Moreover, the positive impact of hypoglycemia treatment recommendations based on the algorithm predictions was demonstrated on in-silico simulations, whereby nocturnal hypoglycemia was reduced by 75.4% (P=0.006) with no negative impact on the overall time in a target glucose range (70-180 mg/dL).

Also described in an SVR model trained on a big dataset and refined using a decision theoretic criterion predicted nocturnal hypoglycemia at bedtime with high sensitivity and specificity reducing risk of nocturnal hypoglycemia. Accurately predicting nocturnal hypoglycemia before a patient goes to sleep may help reduce nighttime hypoglycemia by informing the patient to consume a carbohydrate prior to bedtime.

Additional aspects and advantages will be apparent from the following detailed description of embodiments, which proceeds with reference to the accompanying drawings.

### BRIEF DESCRIPTION OF THE DRAWINGS

To easily identify the discussion of any particular element or act, the most significant digit or digits in a reference number refer to the figure number in which that element is first introduced.

FIG. 1 is a flow diagram showing the SVR algorithm development.

FIG. 2 is a bar chart showing the features used to train the disclosed machine-learning algorithm, ranked by mutual information criterion.

FIG. 3 is a graph showing the ROC curve of the SVR model outlined in FIG. 1 and the selected operating point as compared with the operation point of the direct bedtime glucose heuristic.

FIG. 4 is a graph showing correlation of SVR model predictions of minimum overnight glucose with actual minimum nocturnal glucose values.

FIG. 5 is a flow diagram of process for supporting glucoregulatory management decisions using a decision support recommender to predict hypoglycemic events in an incognizant living subject, in accordance with one embodiment.

FIG. 6 is a block diagram of a computing device, according to one embodiment.

FIG. 7 is a block diagram of a system, according to one embodiment.

### DETAILED DESCRIPTION OF EMBODIMENTS

The following is a set of definitions of terms used in the detailed description of several example scenarios of predicting hypoglycemia in a patient having type 1 diabetes during a period of incognizance carried out in accordance with the disclosed method.

“Actual patient data” are data obtained from a living patient.

“Blood glucose index” is a metric used to quantify the risk of hypo- and hyperglycemia. Examples of blood glucose indicia include the Low Blood Glucose Index (LBGI) and High Blood Glucose Index (HBGI) as reported by Kovatchev et al. (Kovatchev B P, Cox D J, Gonder-Frederick L, Clark W L. Symmetrization of the blood glucose measurement scale and its applications. Diabetes Care. 1997; 20:1655-1658; Kovatchev B P, Cox D J, Gonder-Frederick L, et al. Assessment of risk for severe hypoglycemia among adults with IDDM. Validation of the low blood glucose index. Diabetes Care. 1998; 21:1870-1875; Kovatchev B, Straume M, Cox D, Farhy L. Risk analysis of blood glucose data. A quantitative approach to optimizing the control of insulin dependent diabetes. J Theor Med. 2000; 3:1-10).

“Classifier” is an algorithm that implements classification or the mathematical function implemented by a classification algorithm. “Classification” is a method that identifies to which of a set of categories a new observation belongs based on a set of training data containing observations or instances whose category membership is known. Examples of classifiers that are known in the art include k-nearest neighbor (KNN) classification, Case-based reasoning classification, Decision Tree classification, Naïve Bayes classification, and neural network classification.

“Regression” is a process of measuring the relation between a set of real or continuous values and a set of corresponding real or continuous values. Examples of regression that are well known in the art include linear regression, polynomial regression, SVR, neural network regression, decision tree regression, and random forest regression.

“Validated Patient Data” are a set of data that have been compared to a set of reference data for correctness and completeness.

“Virtual Patient Data” are a set of data created according to a mathematical model of a glucoregulatory system within an in silico virtual patient simulation. Examples include those as reported by Facchinetti et al. and Kovatchev et al. (Facchinetti A, Del Favero S, Sparacino G, Cobelli C. An online failure detection method of the glucose sensor-insulin pump system: improved overnight safety of type-1 diabetic subjects. IEEE transactions on bio-medical engineering. 2013; 60(2):406-16; Kovatchev B P, Breton M, Man C D, Cobelli C. In silico preclinical trials: a proof of concept in closed-loop control of type 1 diabetes. Journal of diabetes science and technology. 2009; 3(1):44-55). Further examples include single and dual hormone virtual patient data as reported by Resalat et al. (Resalat N, El Youssef J, Tyler N, Castle J, Jacobs P G (2019) A statistical virtual patient population for the glucoregulatory system in type 1 diabetes with integrated exercise model. PLoS ONE 14(7):e0217301).

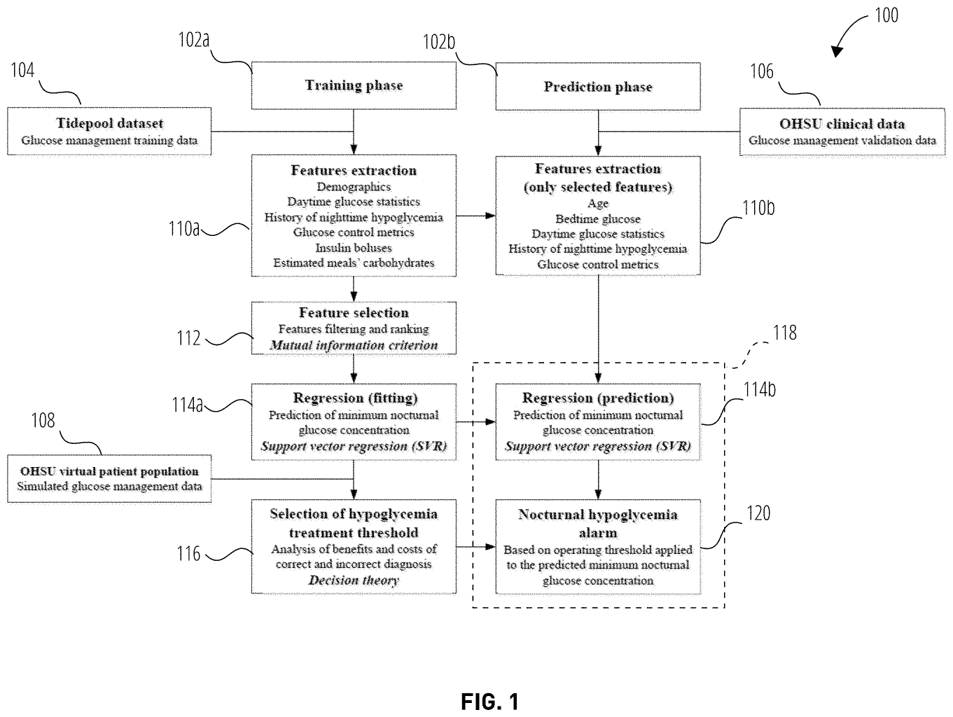

FIG. 1 is an annotated, multi-phase flow diagram 100 showing an example of SVR algorithm development, which entailed both a training phase 102a and a prediction phase 102b, according to operations described below. For completeness, FIG. 1 shows items forming flow diagrams under bother training phase 102a and prediction phase 102b, and inputs of each phase flow inward from its side. Skilled persons will appreciate, however, that some embodiments may implement only a single phase, e.g., prediction phase 102b.

In a left side of flow diagram 100, FIG. 1 shows that a subset of the 4,000+ subjects from a Tidepool Big Data Donation dataset 104 (Tidepool, Palo Alto, Calif., USA) was used to select parameters of the SVR model. Training dataset 104 contained 27,466 days of time-matched CGM and insulin dosed to 124 T1D donors (age 31±19 years, 15±14 years since T1D diagnosis) who are insulin pump users. Data were gathered from multi-vendor CGM and insulin pump devices through the Tidepool.org platform. (Tidepool.org does not provide information about the vendors or models of devices associated with collected data.) CGM readings were obtained every 5 minutes.

Another dataset 106 was used for model validation in prediction phase 102b. Validation dataset 106 was collected during a clinical study in which 10 people with T1D (age 34±6 years, 6F, and 18±10 years since T1D diagnosis) were continuously monitored during a 4-week clinical trial approved by the Institutional Review Board (IRB) at the Oregon Health & Science University (clinicaltrials.gov register NCT02687893). Participants in the validation study identified in Table 1 were evaluated under free-living sensor-augmented insulin-pump therapy. Glucose data were collected every 5 minutes using Dexcom G4 or G4 Share CGM devices (Dexcom Inc., San Diego, Calif., USA), and patients managed their glucose using their own insulin pump.

TABLE 1
Characteristics of participants of study used for model validation.

| | |
|---|---|
| | MEAN ± STD |
| Description | N = 10 subjects |

Demographics

| | |
|---|---|
| Age, years | 33.7 ± 5.8 |
| Female, No. (%) | 6 (60) |
| T1D duration, years | 17.8 ± 10.2 |

Glycemic control

| | |
|---|---|
| Hemoglobin A1c, % | 7.4 ± 1.0 |
| Total daily insulin requirement, U | 41.0 ± 7.3 |
| Estimated average glucose, mg/dL | 164.8 ± 28.3 |
| Validation nights, No. | 115 |
| Nocturnal hypoglycemia events, No. | 38 ± 33 |
| Subjects with 2 or more nocturnal | 8 (80) |
| hypoglycemia events, No. (%) | |

Body composition

| | |
|---|---|
| Body mass, kg | 73.6 ± 9.5 |
| BMI, kg/m² | 24.4 ± 2.1 |

Physical fitness

| | |
|---|---|
| VO₂ max, mL/kg | 46.8 ± 11.6 |
| Resting heart rate, BPM | 62.8 ± 7.8 |

A validated and published virtual patient population 108 was used to optimize the operation point of the SVR algorithm whereby a prediction threshold below which a carbohydrate would be administered to a patient before bedtime was selected. Specifically, 20 virtual patients were generated, using this virtual patient population 108 whereby each virtual patient was matched by weight and total daily insulin requirement to the physiology of an actual patient with T1D. Each virtual patient had a different insulin sensitivity that was statistically sampled from a distribution as described further in Resalat et al. (A statistical virtual patient population for the glucoregulatory system in type 1 diabetes with integrated exercise model, PLoS ONE, 2019). Twenty real-world meal scenarios acquired from people with T1D during a 4-day outpatient study were given to these virtual patients and insulin dosing problems were also imposed. Ten of these virtual patients were used to determine the prediction threshold for the SVR. Validation of the algorithm was done on the remaining 10 virtual patients. Validation was comprised of giving a patient either 15 g of carbohydrate or alternatively a risk-based amount of carbohydrate varying between 15 and 30 g of carbohydrate if nocturnal hypoglycemia was predicted.

FIG. 1 shows that feature extraction 110a entailed a total of 59 features extracted from CGM, insulin, and meal data, as well as demographic variables. Glucose-related features included daytime glucose statistics for different time frames (calculated using data collected between 6:00 AM and 11:00 PM), glucose control metrics, and history of nocturnal glucose concentrations. Insulin on Board and projected Insulin on Board overnight descriptors were also considered, as well as consumed carbohydrates. Details of calculated features are presented below.

Glucose features were calculated across different time frames ranging from 1 to 15 hours before bedtime including statistical descriptions of the measured glucose concentrations (average, standard deviation, coefficient of variation, skewness, and kurtosis), time in hypoglycemia (glucose <70 mg/dL), and time in hyperglycemia (glucose>180 mg/dL). Additional glucose descriptors included bedtime glucose measurements (taken approximately at 11:00 PM), the glucose trend estimated as the slope of a linear model fit to glucose measurements during the 15 minutes preceding 11:00 PM, the average of the minimum nocturnal glucose, and the likelihood of nocturnal hypoglycemia over the seven days prior to the prediction.

Insulin features were represented as inputs to the model as Insulin on Board. Insulin on Board was calculated as a sum of basal insulin and meal boluses given over time with an expected metabolic disposal and normalized by the person's total daily insulin requirement (TDIR). In this work, a simplified model for Insulin on Board calculation was used. It was assumed that the action of rapid-acting insulin lasted approximately 4 hours with a linear decay between the administration time and the 4-hour limit. Features obtained from the normalized insulin data and input into the SVR model included Insulin on Board at bedtime and Insulin on Board projected 4 hours past bedtime.

Meal-related features were estimated by adding the amount of carbohydrates entered by patients into the smart bolus calculator feature of their pumps. Meal sizes during the 6 hours preceding bedtime were considered. Carbohydrate intake data are, however, inherently inaccurate because patients do not always use the bolus calculator wizard to calculate their meal boluses or they might use the calculator to calculate correction boluses.

In feature selection 112, a subset of relevant features is found for a specific learning problem. In some embodiments, predictors for nocturnal hypoglycemia were selected using a relevance criterion called mutual information.

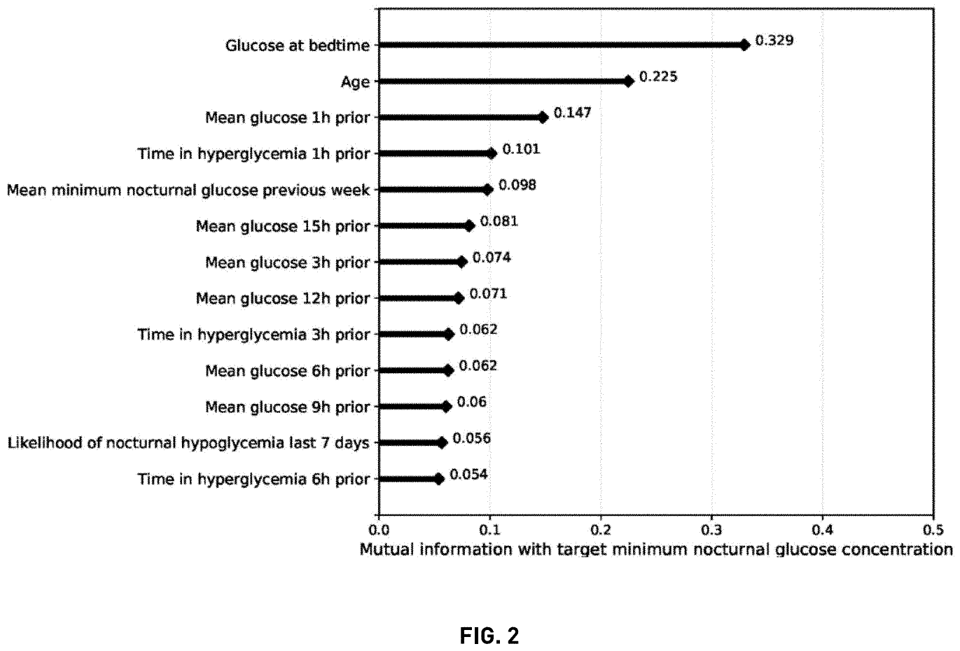

FIG. 2 shows features used to train the disclosed machine-learning algorithm, ranked by the mutual information criterion. Glucose features, particularly bedtime glucose, were the most relevant predictors of the minimum nocturnal glucose concentration. Insulin and meal features were less relevant. This result is consistent with the findings reported by Wilson et al. from a large clinical study in which the factors associated with nocturnal hypoglycemia were retrospectively analyzed in teenagers and young adults with T1D.

With reference to FIG. 1, feature extraction 110b is performed based on feature extraction 110a and feature selection 112 developed during training phase 102a, though applied to dataset 106. A machine learning approach is also shown in hypoglycemia prediction model 114a and hypoglycemia prediction model 114b (also referred to as hypoglycemic event prediction models). In the embodiment of FIG. 1, hypoglycemia prediction model 114b is an SVR model to predict minimum nocturnal glucose level using the 13 selected features extracted from daytime glucose data, previous minimum nocturnal glucose concentration, and likelihood of nocturnal hypoglycemia based on a 7-day history. SVR is trained to find a linear or nonlinear function that maps input features to a target variable constraining the differences between estimated and actual target values to be within an error precision threshold E. E defines an error tolerance margin (E-margin) within which no penalty is associated in the training loss function with points predicted within a distance E from the actual value. For those points predicted outside the E-margin, an error penalty is applied according to the penalty parameter C, which essentially softens the hard margin defined by E. E and C are the hyperparameters of the disclosed model and were tuned through five-fold cross-validation. SVR is a regression technique that has yielded competitive performance in many medical applications including in T1D for short-term prediction of glucose concentrations, though other options for hypoglycemia prediction model 114b are discussed later.

Nocturnal hypoglycemia is defined as any event of any duration in which a patient experienced glucose levels below 70 mg/dL based on CGM readings between 11:00 PM and 6:00 AM. For each patient, for each night in the dataset, a selection of hypoglycemia treatment threshold 116 action (e.g., to announce nocturnal hypoglycemia, or not announce) was chosen to enhance the expected net benefit of the action, given the probability of nocturnal hypoglycemia derived from the SVR model. Gilboa I., Theory of Decision under Uncertainty, Cambridge, N.Y.: Cambridge University Press; 2009 reports that this choice is equivalent to choosing to announce nocturnal hypoglycemia, if and only if the predicted nocturnal minimum of CGM is less than a constant prediction threshold, which is derived using a cost-benefit approach.

Selection of hypoglycemia treatment threshold 116 is, in some embodiments, based on decision theory to select prediction threshold. When deployed for prediction, a decision support recommender 118 includes hypoglycemia prediction model 114b and determines when to warn (e.g., with a nocturnal hypoglycemia alarm 120) a patient of impending nocturnal hypoglycemia so as to inform a decision regarding whether to notify the patient. Decision support recommender 118 considers benefits of giving the recommendation when it is correct as well as the costs of giving an incorrect recommendation. However, there are costs and benefits of not trusting the recommendation and not giving any recommendation at all. This is an example of a type of problem called decision under uncertainty. Under mild conditions, it can be shown that the rational strategy is to choose the action that has the greatest expected net benefit. This decision analytic approach is a formalization of intuitive considerations, which make it possible to expose, discuss, and revise specific assumptions about the problem, and then derive the consequences of those assumptions. A detailed discussion of the methods followed for development of a treatment threshold selection is provided later.

For the problem of recommending a bedtime hypoglycemia treatment, the actions considered are to predict nocturnal hypoglycemia or absence of nocturnal hypoglycemia. Given that nocturnal hypoglycemia might actually be present or actually absent, there are four possible outcomes: (1) predict nocturnal hypoglycemia and nocturnal hypoglycemia is actually present, (2) predict nocturnal hypoglycemia and nocturnal hypoglycemia is actually absent, (3) predict absence of nocturnal hypoglycemia and nocturnal hypoglycemia is actually present, and (4) predict absence of nocturnal hypoglycemia and nocturnal hypoglycemia is actually absent. These outcomes are referred to as true positive (TP), false positive (FP), false negative (FN), and true negative (TN). The benefits of these outcomes are B_{TP}, B_{FP}, B_{FN}, and B_{TN}, respectively.

The probability of nocturnal hypoglycemia p(NH) is derived from the SVR prediction model for minimum nocturnal glucose concentration and its associated error model. The probability of the absence of nocturnal hypoglycemia is therefore

p(-NH)=1−p(NH) Equation 1

Then the expected net benefit (enb) for the action of predicting nocturnal hypoglycemia is

enb(NH)=p(NH)B_{TP}+p(-NH)B_{FP} Equation 2

and the expected net benefit for the action of predicting absence of nocturnal hypoglycemia is

enb(-NH)=p(NH)B_{FN}+p(-NH)B_{TN} Equation 3

Nocturnal hypoglycemia will be predicted if enb(NH)>enb(-NH), which is equivalent to predicting hypoglycemia if

$$p ( N H ) > \frac{( {B}_{T N} - {B}_{F P} )}{( {B}_{TN} - {B}_{F P} + {B}_{T P} - {B}_{F N} )} \qquad Equation 4$$

When there is a large positive reward for high sensitivity (i.e., B_{TP}) or a large negative cost for a missed diagnosis (i.e., B_{FN}), then nocturnal hypoglycemia is predicted for smaller values of p(NH), which is equivalent to raising the prediction threshold for alerting the person that he or she will become hypoglycemic overnight. A similar, complementary conclusion follows from a consideration of B_{TN} and B_{FP}: as those terms become larger, nocturnal hypoglycemia is predicted only for larger values of p(NH) or low cutoff values of the prediction threshold.

Once the critical value of p(NH) is calculated and given a prediction error model, the corresponding minimum nocturnal glucose prediction threshold for alerting the patient can be found by solving for gₜₕ using the Gaussian cumulative distribution function

$$cdf ( x \leq 70 mg \text{/} dL ) = p ( N H ) = 0 . {5}^{*} [ 1 + erf ( \frac{x - ( {g}_{th} + {μ}_{e} )}{\sqrt{2} {σ}_{e}} ) ] \qquad Equation 5$$

where μₑ and σₑ are, respectively, the average and standard deviation of the errors made by the prediction model. The values μₑ=−0.24 mg/dL and σₑ=35.57 mg/dL were estimated from the training dataset.

The benefits B_{TP}, B_{FP}, B_{FN}, and B_{TN} were selected by analyzing the benefits of correct diagnosis of nocturnal hypoglycemia and the cost associated with missed diagnosis and potential overtreatment on 10 virtual subjects during a 5-week in-silico simulation experiment. The LBGI and HBGI were employed to estimate associated costs and benefits on subjects' glucose control using Equation 6:

Bⱼ=(LBGIⁿⁱ−LBGIⁱ)+(HBGIⁿⁱ−HBGIⁱ) Equation 6

where j∈{TF, FP, FN, TN}, superscripts ni and i correspond to the metrics calculated when no intervention is performed and when an intervention is performed based on the predictions of the SVR algorithm for several decision thresholds, respectively. The individual costs and benefits were normalized to account for the imbalance in the number of nocturnal hypoglycemic events vs. the number of nights where subjects did not experience hypoglycemia. The costs and benefits for positive and negative samples were scaled using a class weight calculated as the ratio of the total number of simulated nights for the 10 virtual subjects to twice the number of nights in which subjects experienced nocturnal hypoglycemia (positive class) or the number of nights in which subjects did not experience hypoglycemia (negative class), respectively.

Calculated average values for BTP=10.77, BFP=−2.81, BFN=1.19e-3, and BTN=1.12e-4 resulted in critical p(NH)=0.21, which is equivalent to a prediction threshold of minimum nocturnal glucose concentration of mg/dL.

The decision under uncertainty procedures described above were implemented using 20 virtual patients from the OHSU type 1 diabetes virtual patient population. Ten of the virtual patients and 13,200 nights of data were used to select the prediction thresholds for both the SVR and the direct bedtime glucose heuristic, and 10 of the virtual patients and 3,300 nights of data were used to evaluate the effect of the intervention.

A threshold found for recommending a bedtime hypoglycemia treatment is gₜₕ=99 mg/dL. This means that, if the SVR algorithm predicted that glucose would drop below 99 mg/dL during the night, it would be recommended that the patient consume a carbohydrate at bedtime. The choice of the prediction threshold of 99 mg/dL was intended to enhance the expected net benefit to the patients.

Comparison of the SVR prediction accuracy with a direct heuristic that is oftentimes used to avoid nocturnal hypoglycemia indicates that, if bedtime glucose is below a threshold, the patient should consume a carbohydrate before bed. Calculating the threshold by decision theoretic analysis again using a subset of 10 of the virtual patients reveals that a threshold of 149 mg/dL (gbedtimeₜₕ=149 mg/dL) was appropriate in some embodiments. Using the direct bedtime glucose heuristic, a patient should consume a carbohydrate if the bedtime glucose is less than 149 mg/dL. The SVR algorithm was compared with the direct bedtime glucose heuristic using the evaluation metrics below.

The accuracy of predicting nocturnal hypoglycemia using the AUC, sensitivity, and specificity was evaluated. The 95% confidence interval for the AUC was calculated using the Hanley and McNeil method described in Hanley J A and McNeil B J, The meaning and use of the area under a receiver operating characteristic (ROC) curve, Radiology (1982); 143(1): 29-36. Confusion matrices were generated to characterize the sensitivity and specificity of the prediction algorithms in predicting nocturnal hypoglycemia. The 95% confidence intervals for the sensitivity and specificity of the SVR algorithm and the direct bedtime glucose heuristic at the selected operating threshold were calculated to be the exact Clopper-Pearson confidence intervals described in Clopper C J and Pearson E S, the use of confidence or fiducial limits illustrated in the case of binomial, Biometrika, (1934); 26(4): 404-413.

The accuracy of the SVR prediction algorithm was evaluated by calculating the Pearson correlation between the predicted and actual minimum glucose value during the nighttime hours of 11 PM to 6 AM. The root-mean-square-error between the actual and predicted minimum glucose during the night was estimated. The impact of using either the SVR algorithm or the direct bedtime glucose heuristic on overall glucose control within an in-silico virtual patient population is also reported using percent time in hypoglycemia (<70 mg/dL), percent time in hyperglycemia (>180 mg/dL), and percent time in target range (70-180 mg/dL) as the outcome measures.

Comparison was performed as to the impact of the carbohydrate before bed intervention for both algorithms and with use of an oracle, which had perfect knowledge of when a nocturnal hypoglycemia event would occur. The simulations done with the oracle ensured that a carbohydrate was given to the patient prior to every nocturnal hypoglycemia event and that a carbohydrate was never given if nocturnal hypoglycemia was not indicated. The oracle simulations thereby provide an upper bound on performance above which the prediction algorithms would never exceed.

Table 2 shows SVR performance in predicting nocturnal hypoglycemia on the validation datasets using gₜₕ=99 mg/dL.

TABLE 2

| | Value | |
|---|---|---|
| | Clinical data | Simulated data |
| Performance metric | N = 117 nights | N = 2,706 nights |

Area under the ROC curve for nocturnal hypoglycemia prediction

| | | |
|---|---|---|
| AUC | 0.88 (95% CI, | 0.86 (95% CI, |
| | 0.77-0.99) | 0.83-0.89) |

Nocturnal low glucose excursion prediction
(classification threshold = 70 mg/dL)

| | | |
|---|---|---|
| Sensitivity, % | 94.1 (95% CI, | 82.6 (95% CI, |
| | 71.3-99.9) | 77.4-87.0) |
| Specificity, % | 71.0 (95% CI, | 61.3 (95% CI, |
| | 61.1-79.6) | 59.4-63.3) |
| Correct level 2 nocturnal | 100.0 | 79.7 |
| hypoglycemia detection rate, % | | |
| Over-treated cases (actual | 8.5 | 12.8 |
| minimum glucose >= 99 mg/dL), % | | |

Minimum nocturnal glucose concentration (regression)

| | | |
|---|---|---|
| Pearson correlation | R = 0.73, | R = 0.86, |
| | P < .001 | P < .001 |
| RMSE, mg/dL | 34.5 | 30.8 |

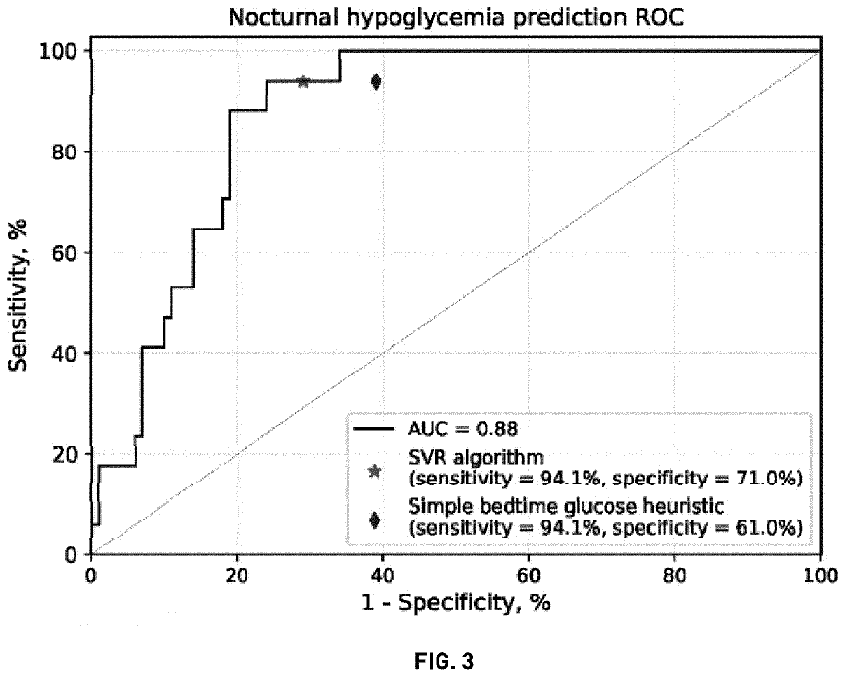

FIG. 3 shows the ROC curve of the disclosed SVR model and the selected operating point as compared with the operation point of the direct bedtime glucose heuristic when validating on the clinical validation dataset using data from nights in which subjects did not eat after 11:00 PM. The SVR prediction model performed better than the direct bedtime glucose heuristic in terms of specificity (71.0% vs. 61.0%) for the same value of sensitivity. The SVR algorithm and the direct bedtime glucose heuristic predicted 94.1% of all nocturnal hypoglycemia events (<70 mg/dL) and all episodes of level 2 hypoglycemia (<54 mg/dL) on data from the validation clinical study.

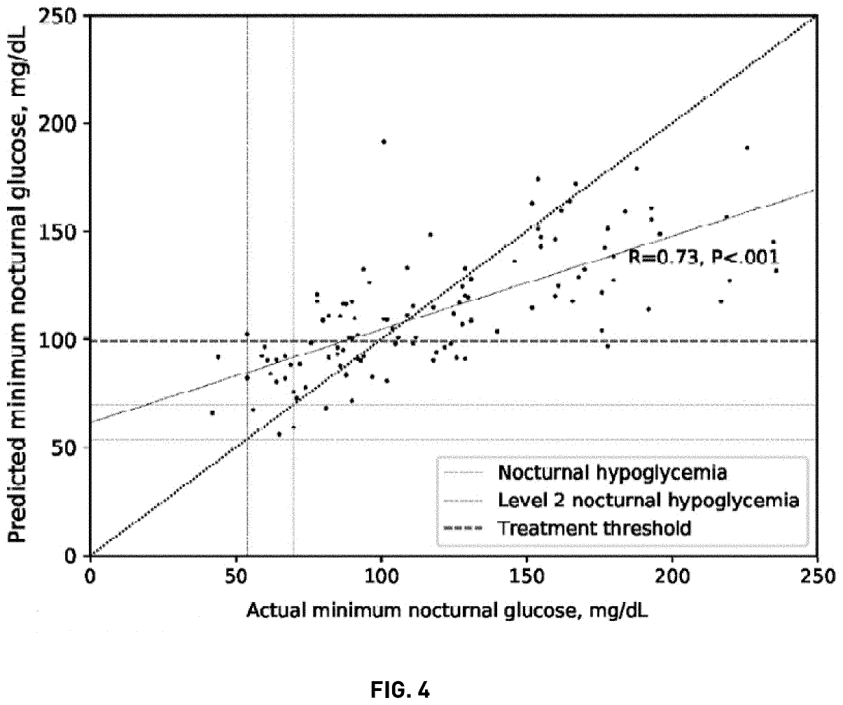

FIG. 4 shows that the SVR model predictions of minimum overnight glucose were well correlated with actual minimum nocturnal glucose values.

Table 3 shows results from validating the impact of recommending either a 15 g or variable 15-30 g carbohydrate bedtime snack based on the predictions of both algorithms for in silico evaluation of the effect of hypoglycemia treatment recommendations at bedtime.

TABLE 3

| Glucose | | | | | | | |
|---|---|---|---|---|---|---|---|
| control | | | | | | | |
| metric | | | | | | | |
| MEAN ± | | | | | | | |
| STD | | | | | | | |
| N = 10 | Intervention | | | | | | |
| subjects | | Bedtime snack 15 g | | | Bedtime snack 15-30 g | | |
| (3,300 | | Oracle- | Bedtime | SVR- | Oracle- | Bedtime | SVR- |
| nights) | None | based | glucose | based | based | glucose | based |

Time in hypoglycemia (<70 mg/dL), %

| | | | | | | | |
|---|---|---|---|---|---|---|---|
| Nighttime | 6.1 ± 5.9 | 2.0 ± 2.8* | 2.3 ± 2.9* | 2.2 ± 2.9* | 0.9 ± 1.6* | 1.6 ± 2.1* | 1.5 ± 2.0* |
| 24 hours | 3.6 ± 2.4 | 1.7 ± 1.1* | 1.8 ± 1.1* | 1.8 ± 1.2* | 1.2 ± 0.7* | 1.5 ± 0.8* | 1.5 ± 0.8* |

Time in hyperglycemia (mg/dL), %

| | | | | | | | |
|---|---|---|---|---|---|---|---|
| Nighttime | 30.2 ± 10.4 | 30.6 ± 10.3 | 36.5 ± 12.2*^{†} | 32.8 ± 10.4* | 31.1 ± 10.0 | 40.0 ± 13.4*^{†} | 33.5 ± 10.5* |
| 24 hours | 36.9 ± 9.9 | 37.2 ± 9.8 | 39.8 ± 9.9*^{†} | 38.2 ± 9.7* ^{†} | 37.4 ± 9.8 | 41.5 ± 9.9* ^{†} | 38.6 ± 9.6* |

Time in range (70-180 mg/dL), %

| | | | | | | | |
|---|---|---|---|---|---|---|---|
| Nighttime | 63.6 ± 6.5 | 67.4 ± 8.5* | 61.2 ± 10.0^{†} | 65.0 ± 8.3^{†} | 68.0 ± 9.1 | 58.4 ± 11.7^{†} | 65.0 ± 9.0 |
| 24 hours | 59.5 ± 9.7 | 61.1 ± 9.2* | 58.3 ± 9.3^{†} | 60.0 ± 9.1^{†} | 61.4 ± 9.2 | 57.0 ± 9.3 ^{†} | 60.0 ± 9.0 |

*Statistically significant difference with respect to no intervention (P < 0.01)
^{†}Statistically significant difference with respect to oracle-based intervention (P < 0.01)

Results in Table 3 are based on data acquired from the 10 simulated T1D patients that were not used to optimize the nocturnal hypoglycemia treatment recommendation threshold across 3,300 nights using real-world meal scenarios. The SVR and direct bedtime glucose heuristic were compared with no intervention, and performance of an oracle-based intervention (i.e., perfect knowledge of nocturnal hypoglycemia) was also evaluated. Without an intervention, the patients spent 6.1%±5.9% time in hypoglycemia overnight. The oracle-based intervention reduced the time in nocturnal hypoglycemia down to 2.0%±2.8% for a fixed 15 g carbohydrate intervention. When the carbohydrate intervention varied between 15 g to 30 g as determined based on hypoglycemia risk, the oracle-based intervention reduced nocturnal hypoglycemia to 0.9%±1.6%. The intervention with variable carbohydrate amount given before bedtime based on the SVR-based algorithm reduced hypoglycemia to 1.5%±2.0% with no statistically significant negative impact on the percent time in target range either overnight or during the 24-hour period after the person went to sleep. While the direct bedtime glucose heuristic with risk-based variable carbohydrate intervention reduced the percent time in nocturnal hypoglycemia to 1.6%±2.1%, there was a substantial amount of over-treatment that resulted in increased time in nocturnal hyperglycemia, whereby the percent time in target range was statistically significantly lower for the direct bedtime glucose heuristic compared with the oracle (58.4%±11.7% vs. 68.0%±9.1%, P<0.001).

The foregoing demonstrates that machine learning methodologies combined with decision uncertainty theoretic analysis can be successfully applied to nocturnal hypoglycemia prediction and treatment. The SVR model trained on a dataset collected from T1D patients under free-living conditions was able to accurately predict the occurrence of nocturnal hypoglycemia with high sensitivity on people with T1D. The positive impact of hypoglycemia treatment recommendations was demonstrated on simulated T1D subjects.

The disclosed SVR algorithm performs better than a direct bedtime glucose heuristic by improving the specificity of the prediction and yielding increased time in target glucose range. However, in the absence of a CGM, smart phone, or other device to acquire the necessary features to run the SVR algorithm, the methods presented above also offer a selected bedtime glucose threshold of 149 mg/dL that may be used by anyone with T1D to make better decisions before bedtime to reduce instances of nocturnal hypoglycemia. The detailed disclosure above shows that 149 mg/dL is a bedtime glucose threshold below which a person with T1D should consume a carbohydrate to reduce the risk of nocturnal hypoglycemia. Both the SVR and direct bedtime glucose heuristic algorithm can have multiple operating points defined by a threshold on the predicted minimum nocturnal glucose concentration such that the sensitivity and specificity can be tuned to match patients' alarm preferences.

The Sakurai et al. article referenced above can be used for comparative analysis. The patients presented in that study were people with type 2 diabetes, compared with T1D in the current paper. Sakurai et al. created a linear regression formula to predict the minimum nocturnal glucose using age, glucose, and insulin features; achieving a root-mean-square error of 31.0 mg/dL. Relevant predictors and reported accuracy were consistent with the findings of our study; however, Sakurai et al. were unable to validate their algorithm in T1D or on a larger data set.

One of the contributions of this disclosure is the in-silico demonstration of the positive effect of recommending a bedtime snack the size of which can be varied based on the minimum glucose predicted by the SVR algorithm or a direct bedtime glucose heuristic. One finding is that a risk-based intervention with a recommended variable-size carbohydrate intake at bedtime can reduce instances of nocturnal hypoglycemia by up to 75.4% with no impact on the overall time in range.

An SVR model designed using decision theory can predict nocturnal hypoglycemia at bedtime with high sensitivity and specificity, accurately identifying 94.1% of nocturnal hypoglycemia events and all level 2 hypoglycemia events. A direct bedtime glucose heuristic (i.e., consume a carbohydrate before bed if glucose is less than 149 mg/dL) with the threshold chosen using decision theory can also predict and help prevent nocturnal hypoglycemia. While it generally entails use of CGM and a computational device, the SVR algorithm yields higher specificity and better glycemic outcomes than those achieved by the direct bedtime glucose heuristic. Big data analytics and machine learning methodologies have the potential to transform diabetes care by providing new ways to effectively prevent complications associated with nocturnal hypoglycemia and improve glycemic control.

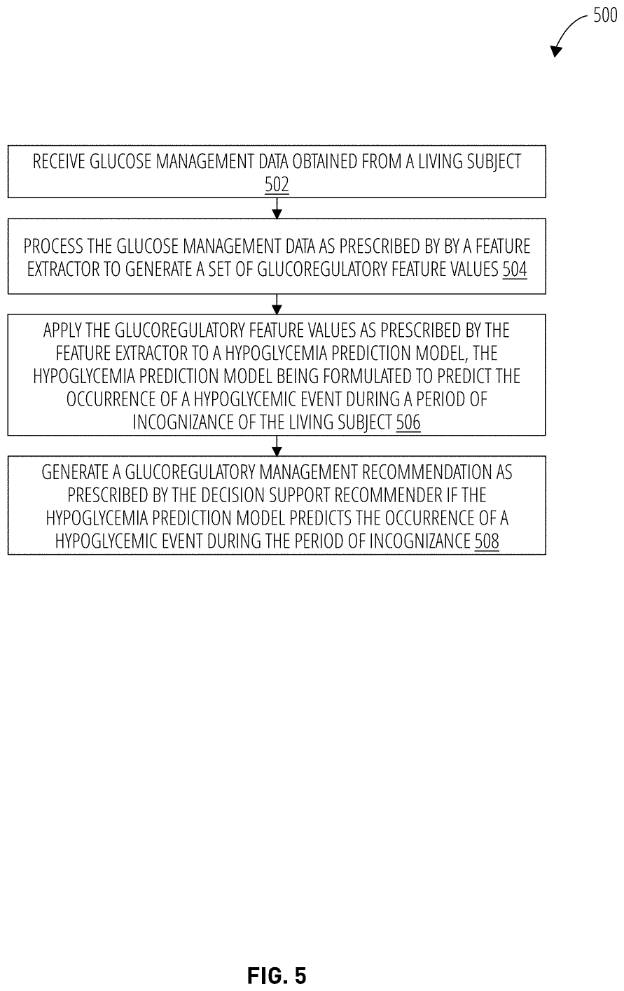

FIG. 5 shows a method 500 for supporting glucoregulatory management decisions using a decision support recommender 118 (FIG. 1) to predict an occurrence of a hypoglycemic event in an incognizant living subject. Initially, method 500 entails receiving 502 glucose management data obtained from a living subject. Glucose management data, including historical insulin therapy, carbohydrate intake, physical activity, and sleep, are obtained from a living subject having type 1 diabetes.

After receiving 502 glucose management data by device having a processor, method 500 entails processing 504 the glucose management data as prescribed by a feature extractor to generate a set of glucoregulatory feature values. In some embodiments, glucoregulatory feature values are selected using a mutual information analysis (see e.g., FIG. 2) and may include a glucose-at-bedtime feature, an age feature, a mean-glucose-1-hr-prior feature, a time-in-hyperglycemia-1 h-prior feature, a mean-minimum-nocturnal-glucose-previous-week feature, a mean-glucose-15-hr-prior feature, mean-glucose-3 hr-prior feature, mean-glucose-12-hr feature, time-in-hyperglycemia-3-hr feature, mean-glucose-6-hr feature, mean-glucose-9-hr feature, a likelihood-of-nocturnal-hypoglycemia-last-7-days feature, and a time-in-hyperglycemia-6-hr feature.

Method 500 then entails applying 5 06 the glucoregulatory feature values as prescribed by the feature extractor to a hypoglycemia prediction model. The hypoglycemia prediction model is formulated to predict the occurrence of a hypoglycemic event during a period of incognizance of the living subject. In some embodiments, the hypoglycemia prediction model of FIG. 5 has been trained using a supervised SVR process that has a hypoglycemic event threshold value and tolerance and regularization hyperparameters.

A hypoglycemic event threshold is a threshold below which hypoglycemia is predicted to occur. Tolerance parameters are used to define stopping criteria for determining when a machine learning algorithm is finished training. And regularization refers to smoothing of the cost function to help prevent over-fitting of the model. The hypoglycemic event threshold, tolerance, and regularization hyperparameters are tuned during the optimization of the machine learning algorithm. The selected tolerance and regularization hyperparameters values are those that give the lowest cross-validation average error from a five-fold cross-validation process. Cross-validation is where a certain portion of the training data is not used for training and is instead used to evaluate accuracy. A five-fold cross validation procedure means that a fifth of the data is held out and not used in training the algorithm, and instead used to evaluate its performance. In five-fold cross validation, different fifths of the data are held out, and then final accuracy is an average of each run. The supervised SVR process trains the hypoglycemia prediction model using both virtual and actual patient data that have been validated as complete and accurate for subjects with type 1 diabetes.

The hypoglycemic event threshold value that is selected according to a decision uncertainty theoretic wherein benefit and risk estimates from the LBGI and HBGI (see Equation 6) are employed to calculate an expected net benefit value for predicting hypoglycemia and an expected net benefit value for predicting the absence of hypoglycemia. In some embodiments, the hypoglycemia threshold value selected is 99 mg/dL based on its correlation to the expected net benefit value for predicting hypoglycemia that is greater than the expected net benefit value for predicting the absence of hypoglycemia.

In some embodiments, hypoglycemia is predicted using the hypoglycemia prediction model whereby glucoregulatory feature values are inputs into the model and the output is a prediction of hypoglycemia. For example, the processed glucoregulatory feature values are applied to the hypoglycemia prediction model to generate an incognizance-minimum glucose value representing the lowest predicted glucose level in mg/dL of the living patient during a period of incognizance, which in this example is a period of sleep. In one example, the hypoglycemia prediction model generates an incognizance-minimum glucose value of 54 mg/dL.

Method 500 then entails generating 508 a glucoregulatory management recommendation as prescribed by the decision support recommender if the hypoglycemia prediction model predicts the occurrence of a hypoglycemic event during the period of incognizance. For instance, in the example where the incognizance-minimum glucose value of 54 mg/dL is predicted, the predicted value of 54 mg/dL is less than the hypoglycemia threshold value selected of 99 mg/dL, the decision support recommender generates a glucoregulatory management recommendation that the living subject consume carbohydrates or adjusts the subject's basal insulin prior to going to bed, thus providing a decision support recommendation for reducing the subject's risk of having a hypoglycemic event during sleep.

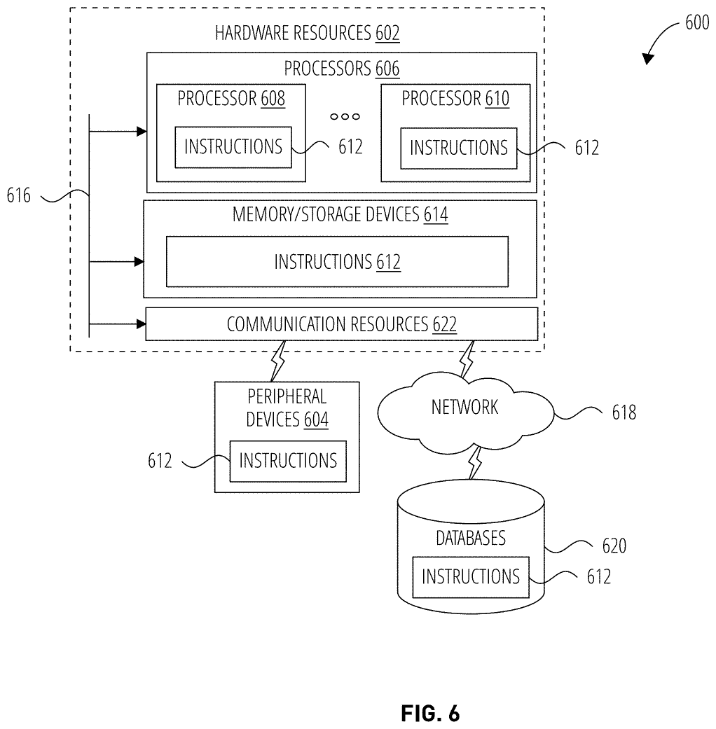

FIG. 6 is a block diagram illustrating components 600, according to some example embodiments, able to read instructions from a machine-readable or computer-readable medium (e.g., a non-transitory machine-readable storage medium), and perform any one or more of the methods discussed herein (e.g., Method 500, FIG. 5). For example, hardware resources 602 may be embodied in a smartwatch, server, tablet computer, or patient-connected device that provides the ability to measure a physiological signal, provide some analysis of that signal, transmit information about that signal, and/or support a user interface to provide information about that signal. This includes an equivalent functional combination, for example a watch that can measure a physiological signal and transmit the data to a computer (including a smartphone), where the computer provides analysis and user interface functions.

Specifically, FIG. 6 shows a diagrammatic representation of hardware resources 602 including one or more processors 606 (or processor cores), one or more memory/storage devices 614, and one or more communication resources 622, each of which may be communicatively coupled via a bus 616.

Processors 606 (e.g., a central processing unit (CPU), a reduced instruction set computing (RISC) processor, a complex instruction set computing (CISC) processor, a graphics processing unit (GPU), a digital signal processor (DSP) such as a baseband processor, an application specific integrated circuit (ASIC), a radio-frequency integrated circuit (RFIC), another processor, or any suitable combination thereof) may include, for example, a processor 608 and a processor 610.

Memory/storage devices 614 may include main memory, disk storage, or any suitable combination thereof. Memory/storage devices 614 may include, but are not limited to any type of volatile or non-volatile memory such as dynamic random access memory (DRAM), static random-access memory (SRAM), erasable programmable read-only memory (EPROM), electrically erasable programmable read-only memory (EEPROM), Flash memory, solid-state storage, etc.

Communication resources 622 may include interconnection or network interface components or other suitable devices to communicate with one or more peripheral devices 604 or one or more databases 620 via a network 618. For example, communication resources 622 may include wired communication components (e.g., for coupling via a Universal Serial Bus (USB)), cellular communication components, NFC components, Bluetooth® components (e.g., Bluetooth® Low Energy), Wi-Fi® components, and other communication components.

Instructions 612 may comprise software, a program, an application, an applet, an app, or other executable code for causing at least any of processors 606 to perform any one or more of the methods discussed herein (e.g., method 500, FIG. 5). In some embodiments, instructions 612 include aspects of decision support recommender 118, FIG. 1) including hypoglycemia prediction model 114b are stored in memory/storage devices 614.

Instructions 612 may reside, completely or partially, within at least one of processors 606 (e.g., within the processor's cache memory), memory/storage devices 614, or any suitable combination thereof. Furthermore, any portion of instructions 612 may be transferred to hardware resources 602 from any combination of the peripheral devices 604 or the databases 620. Accordingly, the memory of processors 606, memory/storage devices 614, peripheral devices 604, and databases 620 are examples of computer-readable and machine-readable media.

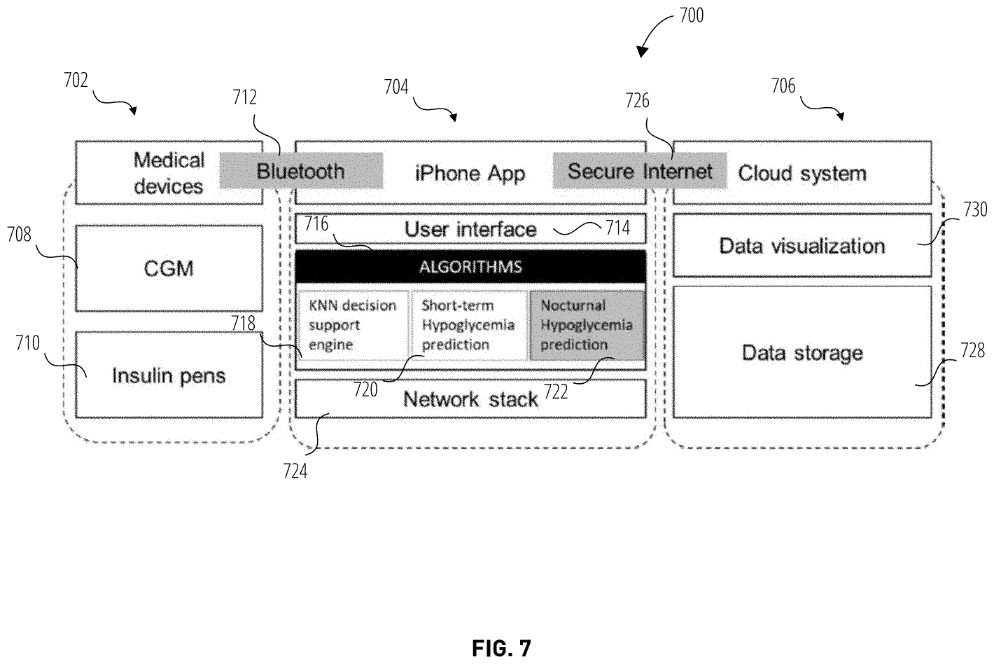

FIG. 7 shows a system 700 for supporting glucoregulatory management decisions using a decision support recommender to predict hypoglycemic events in an incognizant living subject. System 700 includes a medical device 702 (e.g., CGM 708 or insulin pen 710), a user's software application 704 (e.g., an iPhone app, smartwatch app, or other smart device app) running on associated user equipment, and a cloud-based software application 706 running on associated computing devices. Medical device 702 communicate user data over a personal area network (PAN) connection 712 (e.g., Bluetooth) with software application 704.

Software applications 704 generates a user interface 714 that presents to a user nocturnal hypoglycemia alarms 120 (FIG. 1) and associated feedback from decision support recommender 118 (FIG. 1) represented in FIG. 7 as a set of algorithms 716 including KNN decision support engine 718 that can provide the recommendations to the person based on how well that recommendation can improve glycemic outcomes (see Tyler et al. Nature Metabolism 2020), short-term hypoglycemia prediction 720, and nocturnal hypoglycemia prediction 722. For completeness, software applications 704 also shows lower-layer OS components such as network stack 724.

Software application 706 is configured to receive data from software applications 704 through a secure internet connection 726. The data may then be stored in data storage 728 used to generate a data visualization 730 for display on user interface 714.

Skilled persons will appreciate that many changes may be made to the details of the above-described embodiments without departing from the underlying principles of the invention. The scope of the present invention should, therefore, be determined only by claimed inventions and equivalents thereof.

What is claimed is:

1. A method for supporting glucoregulatory management decisions using a decision support recommender to predict an occurrence of a hypoglycemic event in an incognizant living subject, comprising:
receiving glucose management data obtained from a living subject;
processing the glucose management data as prescribed by a feature extractor to generate a set of glucoregulatory feature values;
applying the glucoregulatory feature values as prescribed by the feature extractor to a hypoglycemia prediction model, the hypoglycemia prediction model being formulated to predict the occurrence of a hypoglycemic event during a period of incognizance of the living subject; and
generating a glucoregulatory management recommendation as prescribed by the decision support recommender if the hypoglycemia prediction model predicts the occurrence of a hypoglycemic event during the period of incognizance.

2. The method of claim 1, in which the decision support recommender is trained using a supervised training process.

3. The method of claim 2, in which the supervised training process includes one or both of a regression-based process or a classifier-based process.

4. The method of claim 3, in which the regression-based process is a support vector regression or a neural-network regression.

5. The method of claim 3, in which the classifier-based process is at least one of a random forest process, a decision tree process, and a support vector machine process.

6. The method of claim 2, in which the supervised training process generates a set of hyperparameter values wherein hyperparameter values in the set are optimized by a cross-validation optimization process.

7. The method of claim 6, in which the set of hyperparameter values includes a tolerance hyperparameter and a regularization hyperparameter.

8. The method of claim 1, in which the supervised training process includes at least one of an insulin-metabolism impact model, a glucagon-metabolism impact model, and a nutrient-metabolism impact model.

9. The method of claim 8, in which the nutrient-metabolism impact model includes a carbohydrate model, a protein model, or a fats model.

10. The method of claim 1, in which the supervised training process includes an exercise-metabolism impact model.

11. The method of claim 1, in which the supervised training process includes a stress-metabolism impact model.

12. The method of claim 1, in which the supervised training process includes a sleep-metabolism impact model.

13. The method of claim 1, in which the supervised training process uses virtual patient data, the virtual patient data including single or multiple hormone virtual patient data.

14. The method of claim 13, in which the virtual patient data are generated using a glucoregulatory model.

15. The method of claim 1, in which the supervised training process uses validated or actual patient data.

16. The method of claim 1, in which the hypoglycemia threshold value is selected using a decision under uncertainty theoretic.

17. The method of claim 16, in which the decision under uncertainty theoretic calculates an outcome benefit.

18. The method of claim 17, in which the outcome benefit is calculated using a blood glucose index.

19. The method of claim 16, in which the decision under uncertainty theoretic has first and second expected net benefit values wherein the first and second expected net benefit values represent, respectively, the expected net benefit of predicting hypoglycemia or predicting the absence of hypoglycemia, and the hypoglycemia threshold value correlates with the first expected net benefit value being greater than the second expected net benefit value.

20. The method of claim 16, in which the decision under certainty theoretic is a process comprising:
selecting a B_{TP} value, the B_{TP} value being representative of the benefit of correctly predicting a hypoglycemic event;
selecting a B_{TN} value, the B_{TN} value being representative of the benefit of correctly predicting the absence of a hypoglycemic event;
selecting a B_{FP} value, the B_{FP} value being representative of the benefit of incorrectly predicting a hypoglycemic event;
selecting a B_{FN} value, the B_{TP} value being representative of the benefit of incorrectly predicting the absence of a hypoglycemic event;
calculating a p(IH)Crit value, the p(IH)Crit value representative an expected net benefit for predicting hypoglycemia that is greater than the expected net benefit of predicting the absence of hypoglycemia, wherein: p(IH)Crit>((B_{TN}−B_{TP})/(B_{FP}−B_{FP}+B_{TP}−B_{FN}));
calculating a g_{TH} value wherein p(IH)Crit=0.5×[1+erf(x−(g_{TH}+μₑ)/√2σₑ)] where μₑ and σₑ are the average and standard deviation of errors made by the hypoglycemia prediction model; and
setting the hypoglycemia threshold value equal to the g_{TH}.

21. The method of claim 20, in which the B_{TP}, B_{TN}, B_{FP}, and B_{FN} values are selected using LBGI and HBGI benefit estimates wherein Bⱼ=(LBGIⁿⁱ−LBGIⁱ)+(HBGIⁿⁱ−HBGIⁱ) where j∈{TF, FP, FN, TN}.

22. The method of claim 1, in which the glucose management data includes one or more of the following: historical values related to insulin therapy, glucagon therapy, nutrients, meals, physical activity, and sleep of the living subject.

23. The method of claim 1, in which the glucoregulatory management recommendation is that the living subject ingest an amount of carbohydrate or inject glucagon.

24. The method of claim 23, in which the amount of carbohydrate or amount of glucagon is based on an estimated net benefit calculation.

25. The method of claim 23, in which the amount of carbohydrate or amount of glucagon is based on a linear regression model that correlates the amount of the carbohydrate with a predicted risk of hypoglycemia.
